(19) United States
(12) Patent Application Publication
(10) Pub. No.: US 2025/0114566 A1
(43) Pub. Date: Apr. 10, 2025

(54) SYSTEM FOR ENDOCARDIAL INJECTION

(71) Applicant: Boston Scientific Scimed, Inc., Maple Grove, MN (US)

(72) Inventors: Aiden Flanagan, Kilcolgan (IE); Martyn G. Folan, Loughrea (IE); Tim O'Connor, Galway (IE); Jonathan Heelan, Limerick (IE); Martin Lawrence Fawdry, Galway (IE); Michael Hughes, Claregalway (IE); Michael James Kelly, Galway (IE)

(73) Assignee: Boston Scientific Scimed, Inc., Maple Grove, MN (US)

(21) Appl. No.: 18/905,233

(22) Filed: Oct. 3, 2024

Related U.S. Application Data
(60) Provisional application No. 63/542,441, filed on Oct. 4, 2023.

(51) Int. Cl. A61M 25/01; A61B 8/12; A61B 18/00; A61B 18/14; A61M 25/00

(52) U.S. Cl. CPC A61M 25/0133; A61B 8/12; A61B 18/1477; A61M 25/0084; A61B 2018/00577; A61B 2018/00595; A61M 2205/3606

(57) ABSTRACT

System for endocardial injection are disclosed. An example system may include a first steerable catheter having a first lumen formed therein. A second steerable catheter may be disposed within the first lumen. The second steerable catheter may have a second lumen formed therein. An injection catheter may be disposed within the second lumen. The injection catheter may have a distal end region. The distal end region may include an imaging section and a needle section. The system may include an imaging device configured to be disposed within the imaging section.

### CROSS-REFERENCE TO RELATED APPLICATIONS

This application claims the benefit of priority under 35 U.S.C. § 119 of U.S. Provisional Application No. 63/542,441, filed Oct. 4, 2023, the entire disclosure of which is hereby incorporated by reference.

### TECHNICAL FIELD

The present disclosure pertains to medical devices, and methods for manufacturing medical devices. More particularly, the present disclosure pertains to systems for endocardial injection.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for endocardial injection is disclosed. The system comprises: a first steerable catheter having a first lumen formed therein; a second steerable catheter disposed within the first lumen, the second steerable catheter having a second lumen formed therein; an injection catheter disposed within the second lumen, the injection catheter having a distal end region; wherein the distal end region includes an imaging section and a needle section; and an imaging device configured to be disposed within the imaging section.

Alternatively or additionally to any of the embodiments above, the first steerable catheter is steerable in at least one plane.

Alternatively or additionally to any of the embodiments above, the first steerable catheter is steerable in two planes.

Alternatively or additionally to any of the embodiments above, the second steerable catheter is steerable in two planes.

Alternatively or additionally to any of the embodiments above, the imaging section and the needle section are arranged in a side-by-side arrangement.

Alternatively or additionally to any of the embodiments above, the needle section has a fluid injection lumen formed therein.

Alternatively or additionally to any of the embodiments above, the imaging device includes an intravascular ultrasound imaging transducer.

Alternatively or additionally to any of the embodiments above, the imaging device is slidably disposed within the injection catheter.

Alternatively or additionally to any of the embodiments above, further comprising a third steerable catheter having a third lumen formed therein, wherein the first steerable catheter is disposed within the third lumen.

Alternatively or additionally to any of the embodiments above, the needle section includes a heating element.

Alternatively or additionally to any of the embodiments above, the needle section includes a cooling element.

Alternatively or additionally to any of the embodiments above, the needle section is configured to deliver electrocautery current to tissue.

Alternatively or additionally to any of the embodiments above, the needle section is configured to deliver a therapeutic gel to endocardial tissue.

A system for endocardial injection is disclosed. The system comprises: a steerable catheter assembly including two or more steerable catheters nested together; an injection catheter disposed within the steerable catheter assembly, the injection catheter being configured to a therapeutic gel to endocardial tissue; wherein the injection catheter has a distal end region with an imaging section and a needle section disposed adjacent to the imaging section; an imaging device slidably disposed within the injection catheter; and wherein the imaging device includes an ultrasound transducer configured to be disposed along the imaging section.

Alternatively or additionally to any of the embodiments above, the steerable catheter assembly includes at least one steerable catheter that is steerable in a single plane.

Alternatively or additionally to any of the embodiments above, the steerable catheter assembly includes at least one steerable catheter that is steerable in two planes.

Alternatively or additionally to any of the embodiments above, the needle section includes a heating element.

Alternatively or additionally to any of the embodiments above, the needle section includes a cooling element.

Alternatively or additionally to any of the embodiments above, the needle section is configured to deliver electrocautery current to tissue.

A method for delivering a therapeutic gel to endocardial tissue is disclosed. The method comprises: navigating a endocardial injection system through a body lumen to a target location, the endocardial injection system comprising: a first steerable catheter having a first lumen formed therein, a second steerable catheter disposed within the first lumen, the second steerable catheter having a second lumen formed therein, an injection catheter disposed within the second lumen, the injection catheter having a distal end region, wherein the distal end region includes an imaging section and a needle section, and an imaging device configured to be disposed within the imaging section; wherein navigating the endocardial injection system through the body lumen to the target location includes steering the first steerable catheter, steering the second steerable catheter, or steering both the first steerable catheter and the second steerable catheter; engaging the target location with the needle section; and delivering the therapeutic gel through the needle section and into the target location.

A system for endocardial injection is disclosed. The system comprises: one or more steerable catheters; an injection catheter disposed within a lumen of at least one of the one or more steerable catheters, the injection catheter having a distal end region; wherein the distal end region includes an imaging section and a needle section; and an imaging device configured to be disposed within the imaging section.

Alternatively or additionally to any of the embodiments above, the one or more steerable catheters include a first steerable catheter having a first lumen formed therein and a second steerable catheter disposed within the first lumen.

Alternatively or additionally to any of the embodiments above, the second steerable catheter has a second lumen formed therein.

Alternatively or additionally to any of the embodiments above; the injection catheter is disposed within the second lumen.

Alternatively or additionally to any of the embodiments above, the first steerable catheter is steerable in at least one plane.

Alternatively or additionally to any of the embodiments above, the first steerable catheter is steerable in two planes.

Alternatively or additionally to any of the embodiments above, the second steerable catheter is steerable in two planes.

Alternatively or additionally to any of the embodiments above, the imaging section and the needle section are arranged in a side-by-side arrangement.

Alternatively or additionally to any of the embodiments above, the needle section has a fluid injection lumen formed therein.

Alternatively or additionally to any of the embodiments above, the imaging device includes an intravascular ultrasound imaging transducer.

Alternatively or additionally to any of the embodiments above, the imaging device is slidably disposed within the injection catheter.

Alternatively or additionally to any of the embodiments above, further comprising a third steerable catheter having a third lumen formed therein, wherein the first steerable catheter is disposed within the third lumen.

Alternatively or additionally to any of the embodiments above, the needle section includes a heating element.

Alternatively or additionally to any of the embodiments above, the needle section includes a cooling element.

Alternatively or additionally to any of the embodiments above, the needle section is configured to deliver electrocautery current to tissue.

Alternatively or additionally to any of the embodiments above, the needle section is configured to deliver a therapeutic gel to endocardial tissue.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan overview of an example system for endocardial injection.

FIG. 2 is a schematic depiction of an example steerable catheter.

FIG. 3 is a schematic depiction of an example steerable catheter.

FIG. 4 is a plan view of a portion of an example system for endocardial injection.

FIG. 5 is a side view of a portion of an example system for endocardial injection.

FIG. 6 is a side view of a portion of an example system for endocardial injection.

FIG. 7 is a plan view of a portion of an example system for endocardial injection.

FIG. 8 is a side view of a portion of an example system for endocardial injection.

FIG. 9 is a plan view of a portion of an example system for endocardial injection.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

A therapeutic gel may be used to help reduce and/or prevent heart failure after a myocardial infarction (MI). For example, a clinician may inject a therapeutic gel into the endocardium of a patient after MI, for example about 5-7 days after MI. The endocardial gel injection may help to improve ischemic myocardial tissue recover with less fibrosis and increased angiogenesis and cardiac function. It can be appreciated that it may be technically challenging to reach an endocardial injection site, penetrate a needle at the injection site, and to make multiple injections (e.g., when desired/needed) while delivering the desired quantity of gel and while reducing/preventing back flow of the gel into the heart (e.g., the left ventricle). Disclosed herein are systems for endocardial injection, for example of a therapeutic gel after MI. The systems disclosed herein may overcome at least some of the technical challenges associated with endocardial injections. Some of the details of such systems are disclosed herein.

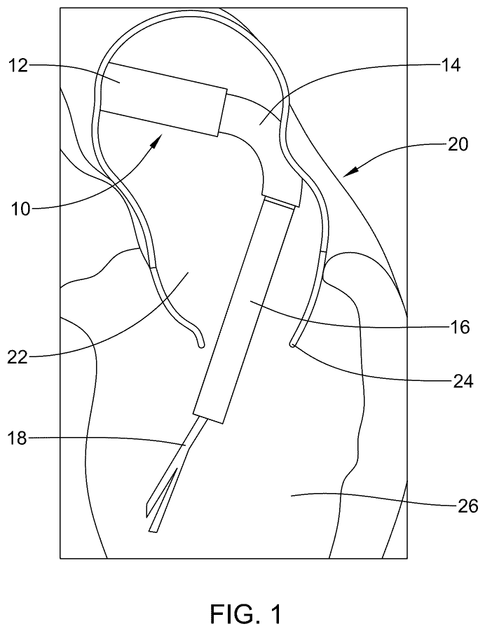

FIG. 1 illustrates an example system 10 for endocardial injection. The system 10 may include one or more steerable catheters such as a first steerable catheter 12, a second steerable catheter 14, and a third steerable catheter 16. In this example, the steerable catheters 12, 14, 16 are depicted as being nested and/or otherwise arranged so that the outermost catheter (e.g., in this example the outermost catheter is the first steerable catheter 12) defines a lumen. The next catheter inward or middle catheter (e.g., in this example the next catheter inward is the second steerable catheter 14) is disposed within the lumen. The middle catheter (e.g., in this example the second steerable catheter 14) defines a lumen and the innermost catheter (e.g., in this example the innermost catheter is the third steerable catheter 16) is disposed within the lumen. It can be appreciated that the numbering (e.g., first, second, and/or third steerable catheters 12, 14, 16) is not intended to be limiting and is not intended to be indicative of the arrangement or positioning of the steerable catheters 12, 14, 16. For example, in some instances the first steerable catheter 12 may be a middle catheter, an innermost catheter, or have any other suitable arrangement/position. The same is true of the other steerable catheters. Furthermore, some systems contemplated may include only a single steerable catheter, two or more steerable catheters, one or more “non-steerable” catheter intermixed with steerable catheters, and/or the like

An injection catheter 18 may be disposed within one or more of the catheters 12, 14, 16. In this example, the injection catheter 18 may be disposed within the third steerable catheter 16 (e.g., within a lumen of third steerable catheter 16). In systems where less than three steerable catheters (e.g., two steerable catheters are utilized, and/or a combination of steerable and non-steerable catheters are utilized), the injection catheter 18 may be disposed within the lumen of one of the catheters of such a system. The injection catheter 18, in general, may be configured to deliver a therapeutic substance to a target region. For example, the injection catheter 18 may be configured to deliver a therapeutic gel

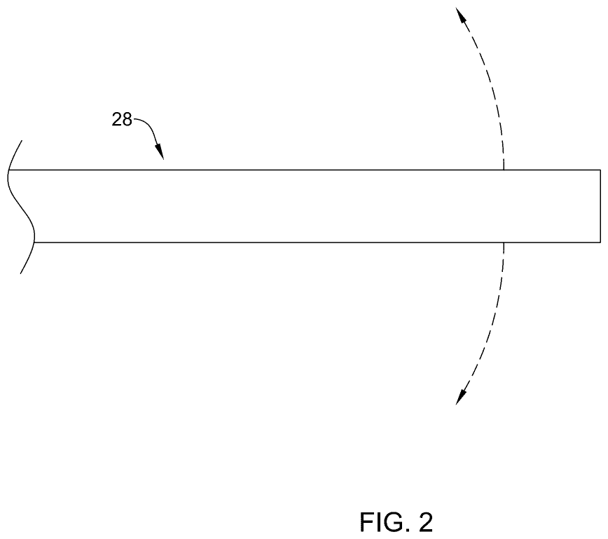

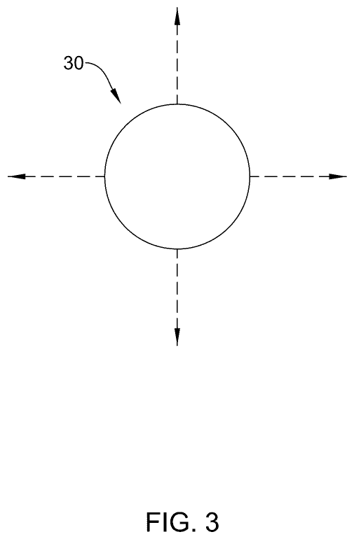

In use, the system 10 may be navigated into a heart 20. When doing so, the system may extend into a left atrium 22, through the mitral valve 24, and into the left ventricle 26. This may include advancing the system through the vena cava, performing a transseptal crossing through the atrial septum and into the left atrium 22. Other navigation methodologies are contemplated. As the names suggest, to help facilitate navigation, one or more of the catheters 12, 14, 16 may be steerable. This may include catheters steerable in at least one plane (e.g., yaw or pitch) and/or catheter steerable in at least two planes (e.g., yaw and pitch). For example, FIG. 2 schematically depicts a catheter 28, which may be representative of one or more of the steerable catheters 12, 14, 16, that is steerable in one plane. FIG. 3 schematically depicts a catheter 30, which may be representative of one or more of the catheters 12, 14, 16, that is steerable in two planes. FIGS. 2-3 are meant to depict that the steerable catheters 12, 14, 16 may be steerable in at least one plane or the steerable catheters 12, 14, 16 may be steerable in at least two planes. The structure of the steerable catheters 12, 14, 16 may vary to include pull wires, articulating structures/regions, and/or other structural features that help to allow the steerable catheters 12, 14, 16 to be navigated/steered.

It can be appreciated that navigating the system 10 to the heart 20 can include a trans-septal approach. Other approaches may be utilized including retrograde approaches where a trans-femoral and/or aortic approach is utilized. For example, the system 10 may be advanced through a femoral artery, through the aorta, and into the left ventricle.

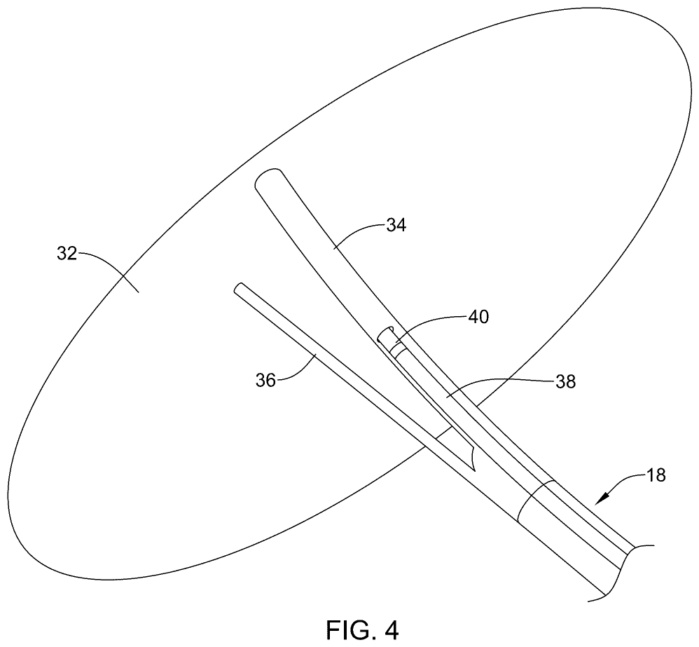

FIG. 4 illustrates the distal end region of the injection catheter 18 disposed at and/or engaged with a target region 32. Here it can be seen that a distal end region of the injection catheter 18 may include a first or imaging section 34 and a second or needle section 36. In some instances, the first section 34 may include or be termed an anchoring section. In some instances, the injection catheter 18 may be similar in form and function to the iNod™ Ultrasound Guided System, commercially available from Boston Scientific.

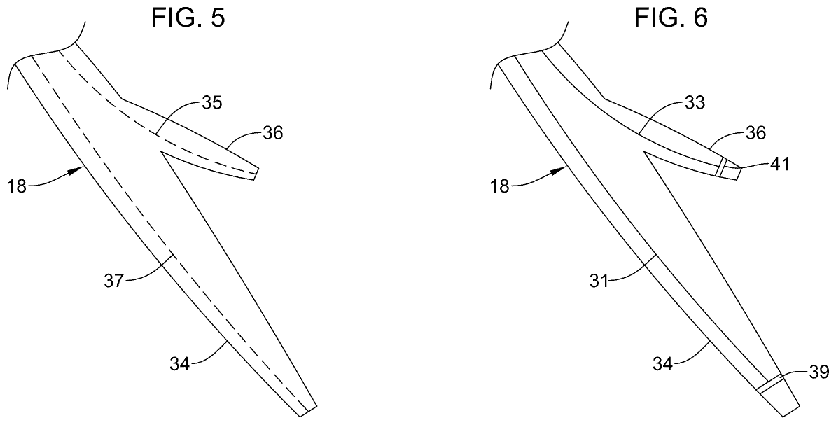

An imaging device 38 may be disposed within the injection catheter 18 and may be configured to extend along the imaging section 34. The imaging device 38 may include an imaging transducer 40 such as an ultrasound transducer. The imaging device 38 may be axially fixed within the imaging section 34. Alternatively, the imaging device 38 may be slidable within the imaging section 34. For example, the imaging device 38 may be disposed within a lumen (e.g., the lumen 37 schematically depicted in FIG. 5) formed in the injection catheter 18. In some instances, the imaging device 38 may extend all the way to the proximal end of the injection catheter 18 and be coupled to a handle or actuator (not shown) that may be used to control the position of the imaging device 38 within the injection catheter 18 (and/or the imaging section 34). Using axial translation and rotation of the imaging device 38, a three-dimensional cylindrical view can be built up, for example showing tissue/anatomy near/adjacent to the target region 32. This may allow the whole needle/needle section 36 to be observed during tissue penetration and/or injection of a therapeutic substance or gel. It can be appreciated that the imaging device 38 may be coupled to suitable hardware sufficiently to energize and/or activate the imaging transducer 40. The imaging device 38 may also be coupled to suitable control and/or display hardware sufficiently to display of anatomy imaged by the imaging device 38.

The needle section 36 may be used to pierce the target region 32. In some instances, the needle section 36 may take the form of or otherwise include a needle. In some of these instances, the needle/needle section 36 may be shiftable and/or retractable relative to the injection catheter 18. For example, the needle/needle section 36 may be retracted so that the end of the needle/needle section 36 is disposed within the injection catheter (e.g., disposed within the lumen 35 schematically depicted in FIG. 5). Upon reaching the target region 32, the needle/needle section 36 may be advanced into contact with the target region 32. When suitably positioned, a therapeutic substance or gel may be injected into the target region 32 through the needle/needle section 36. In some instances, the needle/needle section 36 may be withdrawn while injecting the therapeutic substance into the target region 32.

In some instances, it may be desirable to withdraw the needle/needle section 36 from the target region 32 at a desired rate. The withdraw rate may be a function of the cross-sectional area of the needle/needle section 36 and the injection velocity. For example, it may be desirable to withdraw the needle/needle section 36 at a rate equal to:

$$Withdrawl Rate = - C \frac{\dot{V}}{A}$$

where C is a constant, \dot{V} is the volumetric flow-rate, and A is the cross-sectional area of the needle/needle section 36.
It may be desirable to withdraw the needle at a rate slightly less than the injection velocity so that a desired injection pressure is maintained driving gel penetration into the parenchyma. The injection velocity may be determined by syringe/injection parameters.

Other control algorithms can be used optimize needle penetration depth. Such algorithms may utilize parameters such as myocardial depth (e.g., as measured by the imaging device 38), needle/needle section 36 depth as measured by an external scale on the needle and/or an echogenic marker, the angle of the needle penetration in YAW and PITCH as measured by the imaging device 38. A suitable control and/or processing unit may be used in conjunction with the system 10 to facilitate visualization of needle/needle section 36 depth/penetration and/or other suitable parameters.

It can be appreciated that the imaging device 38 can be used in conjunction with the needle section 36 in order to guide/navigate the system 10 in a manner that aid delivery of the therapeutic substance to the desired location at/adjacent to the target region 32. For example, the imaging transducer 40 can be activated to image during navigation of the system 10 toward the target region 32, during actuation of the needle section 36 (e.g., when utilized), during engagement of the imaging section 34 and/or the needle section 36 with the target region 32, and/or during the infusion/injection of the therapeutic substance into the target region 32. This may include imaging across 360 degrees and/or stitching together multiple images in order to visualize injection boluses. In at least some instances, imaging may be able to discern the therapeutic substance (e.g., discern between the therapeutic substance and tissue) so that injection can be stopped if backflow is observed.

The therapeutic substance may include a material such as a non-reversible thermosensitive gel. On example of a non-reversible thermosensitive gel that may be used is an ELR-SCAR gel. Such a substance may be a liquid at cooler temperatures (e.g., at about 17 degrees C. or lower) and may transform to a gel at higher temperatures including around body temperature (e.g., 37 degrees C.). Other materials contemplated include conformable embolic and/or sheer thinning materials such as OBSIOD™ Conformable Embolic material (commercially available from Boston Scientific), ORISE™ gel (commercially available from Boston Scientific), hyaluronic acid gel, reverse thermosensitive gels such as BACKSTOP gel (commercially available from Boston Scientific), combinations thereof, and/or the like In some instances, the therapeutic substance may include gas microbubbles, which may help to visualize the therapeutic substance during/after injection.

FIGS. 5-6, which are highly schematic in nature, illustrate some of the structures that may make up and/or otherwise be included with the injection catheter 18. For example, FIG. 5 depicts that the injection catheter 18 may include one or more lumens such as a lumen 37 in fluid communication with the imaging section 34 and/or a lumen 35 in fluid communication with the needle section 36. As indicated above, the lumen 37 may be used to house the imaging device 38. The lumen 35 may be used during the infusion/injection of the therapeutic substance. In this example, the lumens 35, 37 are represented by dashed lines. The shape, path through the injection catheter 18, arrangement of the lumens 35, 37 within the injection catheter 18, combinations thereof, and/or the like can vary.

In some instances, one or both of the lumens 35, 37 may be used to pass a cooling fluid (e.g., such as cooled saline) and/or gas down the injection catheter 18. In cases where the therapeutic substance used with the system 10 is ELR-SCAR, the passing of cooling fluid down the injection catheter 18 may help to maintain the therapeutic substance in a desired stated (e.g., liquid). Lumens in one or more of the steerable catheters 12, 14, 16 may also be used to help keep the therapeutic substance at a desired temperature and/or state. Flow through the lumens 35, 37 and/or through the lumens in one or more of the steerable catheters 12, 14, 16 may set at about 1-50 ml/minute, or at about 5-30 ml/minute, or at about 5-10ml/minutes, or at about 30 ml/minute. These flow rates may be for system where flow is either through the end of the injection catheter 18 (e.g., an open flow system) or in a recirculation system within the injection catheter 18. These are just examples. In instances where a cooling fluid such as a cooling gas is utilized, a suction/vacuum device can be used to generate negative pressure. Negative pressure at the distal end region of the injection catheter 18 may help to stabilize and/or hold the injection catheter 18 at the target region 32.

FIG. 6 illustrates that the injection catheter 18 may include one or more sensors and/or heating/cooling elements. For example, a first sensor and/or heating/cooling element 39 may be disposed along the imaging section 34. The first sensor and/or heating/cooling element 39 may take the form of a temperature sensor. The temperature sensor may help monitor the temperature along the injection catheter 18 so as, for example, to maintain the therapeutic substance at the desired temperature. If the temperature is determined to be different from a target temperature, circulation of a cooling fluid (e.g., cooled saline) through the injection catheter 18 may be modulated.

In some of these and in other instances, the first sensor and/or heating/cooling element 39 may take the form of or include a cooling element (e.g., a cryogenic cooling element). In some instances, the cooling element may take the form of a cooling coil such as a dual wire, wrapped, helical structure configured to encourage continuous travel of a cooling fluid therethrough. It can be appreciated that a cooling element may help to slow the gelling of the therapeutic substance (e.g., when the gelling substances is an ELR-SCAR gel). In some of these and in other instances, the first sensor and/or heating/cooling element 39 may take the form of or include a heating element. It can be appreciated that a heating element may help to accelerate the gelling of the therapeutic substance (e.g., when the gelling substances is an ELR-SCAR gel). In some of these and in other instances, the first sensor and/or heating/cooling element 39 may take the form of or include an electrocautery member that may be used, for example, to aid penetration of fibrotic tissue and/or ablate tissue (e.g., which may loosen damaged tissue and/or facilitate injection).

A second sensor and/or heating/cooling element 41 may be disposed along the needle section 36. The second sensor and/or heating/cooling element 41 may take the form of a temperature sensor. The temperature sensor may help monitor the temperature along the injection catheter 18 so as, for example, to maintain the therapeutic substance at the desired temperature. If the temperature is determined to be different from a target temperature, circulation of a cooling fluid (e.g., cooled saline) through the injection catheter 18 may be modulated. In some of these and in other instances, the second sensor and/or heating/cooling element 41 may take the form of or include a heating element. In some of these and in other instances, the second sensor and/or heating/cooling element 41 may take the form of or include a cooling element (e.g., a cryogenic cooling element). In some instances, the cooling element may take the form of a cooling coil such as a dual wire, wrapped, helical structure configured to encourage continuous travel of a cooling fluid therethrough. It can be appreciated that a cooling element may help to slow the gelling of the therapeutic substance (e.g., when the gelling substances is an ELR-SCAR gel). In some of these and in other instances, the second sensor and/or heating/cooling element 41 may take the form of or include a heating element. It can be appreciated that a heating element may help to accelerate the gelling of the therapeutic substance (e.g., when the gelling substances is an ELR-SCAR gel). In some of these and in other instances, the second sensor and/or heating/cooling element 41 may take the form of or include an electrocautery member that may be used, for example, to aid penetration of fibrotic tissue and/or ablate tissue (e.g., which may loosen damaged tissue and/or facilitate injection).

In some instances, the first sensor and/or heating/cooling element 39 and/or the second sensor and/or heating/cooling element 41 may include an echogenic marker. For example, an echogenic marker may allow a clinician to monitor the penetration depth of the needle/needle section 36 while imaging with the imaging device 38. For example, the echogenic marker can be disposed along the needle/needle section 36 at or adjacent to a desired optimum distance from the distal tip of the needle/needle section 36. Injection of the therapeutic substance can be stopped when the needle/needle section 36 is withdrawn a distance (e.g., which may include when the echogenic marker becomes visible). In some of these and in other instances, the first and/or second sensor and/or heating/cooling elements 39, 41 may include a magnetic sensor that can help to facilitate determining the position of the system (e.g., using an externally generated magnetic field).

A first heating/cooling member 31 may be disposed along the imaging section 34. The first heating/cooling member 31 may take the form of a cooling rod disposed within or along the lumen 37. In some instances, the cooling rod may be secured/fixed within the injection catheter 18. Alternatively, the cooling rod may be removable and/or replaceable within the injection catheter 18. In other instances, the first heating/cooling member 31 may take the form of a heating rod.

A second heating/cooling member 33 may be disposed along the needle section 36. The second heating/cooling member 33 may take the form of a cooling rod disposed within or along the lumen 35. In some instances, the cooling rod may be secured/fixed within the injection catheter 18. Alternatively, the cooling rod may be removable and/or replaceable within the injection catheter 18. In other instances, the second heating/cooling member 33 may take the form of a heating rod.

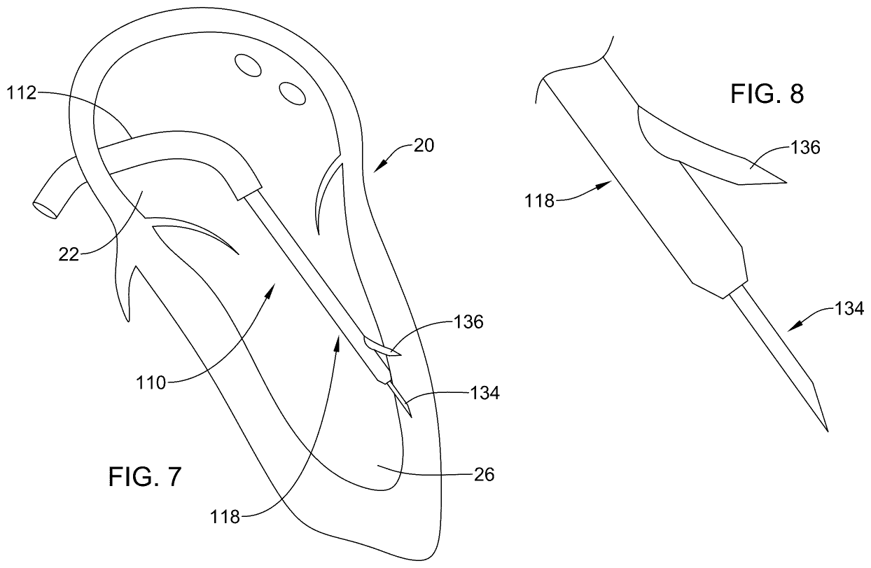

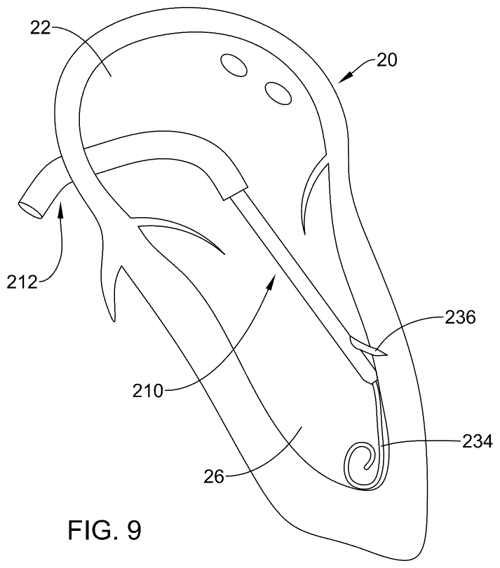

FIGS. 7-8 illustrate another example system 110 that may be similar in form and function to other systems disclosed herein. For example, the system 110 may include one or more steerable catheters, represented in FIG. 7 by reference number 112, and an injection catheter 118. The injection catheter 118 may include first or stabilizing section 134 and a second or injection section 136. In this example, the stabilizing section 134 may take the form of a needle or tissue-engaging member that is configured to engage target tissues in order to facilitate stabilization and/or holding the position of the system 110. For example, the stabilizing section 134 may be used to pierce target tissue adjacent to a target location so that the injection section 136 can be used to inject a therapeutic substance.

In some instances, the stabilizing section 134 may be retractable/advanceable. In other words, the stabilizing section 134 may be configured to shift between a first or delivery configuration (e.g., where the stabilizing section 134 is retracted into the injection catheter 118) and a second or stabilizing configuration (e.g., where the stabilizing section 134 is advanced out of the injection catheter 118 so as to allow/effect engagement with target tissue). The injection section 136, which may resemble other injection sections disclosed herein, may be similarly shiftable between a first or delivery configuration (e.g., where the injection section 136 is retracted into the injection catheter 118) and a second or injection configuration (e.g., where the injection section 136 is advanced out of the injection catheter 118 so as to allow/effect engagement with target tissue).

FIG. 9 illustrates another example system 210 that may be similar in form and function to other systems disclosed herein. For example, the system 210 may include one or more steerable catheters, represented in FIG. 9 by reference number 212, and an injection catheter 218. The injection catheter 218 may include first or stabilizing section 234 and a second or injection section 236. In this example, the stabilizing section 234 may take the form of a pigtail or similar catheter section that is configured to engage a target region in order to facilitate stabilization and/or holding the position of the system 210. For example, the stabilizing section 234 may be seated within the left ventricle so that the injection section 236 can be used to inject a therapeutic substance.

In some instances, the stabilizing section 234 may be retractable/advanceable. In other words, the stabilizing section 234 may be configured to shift between a first or delivery configuration (e.g., where the stabilizing section 234 is retracted into the injection catheter 218) and a second or stabilizing configuration (e.g., where the stabilizing section 234 is advanced out of the injection catheter 118 so as to allow/effect engagement with target tissue, for example in the left ventricle). The injection section 236, which may resemble other injection sections disclosed herein, may be similarly shiftable between a first or delivery configuration (e.g., where the injection section 236 is retracted into the injection catheter 218) and a second or injection configuration (e.g., where the injection section 236 is advanced out of the injection catheter 218 so as to allow/effect engagement with target tissue).

The materials that can be used for the various components of the system 10 (and/or other systems disclosed herein) may include those commonly associated with medical devices. For simplicity purposes, the following discussion makes reference to the catheter 12 and other components of the system 10. However, this is not intended to limit the devices and methods described herein, as the discussion may be applied to other similar tubular members and/or components of tubular members or devices disclosed herein.

The catheter 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for endocardial injection, the system comprising:
a first steerable catheter having a first lumen formed therein;
a second steerable catheter disposed within the first lumen, the second steerable catheter having a second lumen formed therein;
an injection catheter disposed within the second lumen, the injection catheter having a distal end region;
wherein the distal end region includes an imaging section and a needle section; and
an imaging device configured to be disposed within the imaging section.

2. The system of claim 1, wherein the first steerable catheter is steerable in at least one plane.

3. The system of claim 1, wherein the first steerable catheter is steerable in two planes.

4. The system of claim 1, wherein the second steerable catheter is steerable in two planes.

5. The system of claim 1, wherein the imaging section and the needle section are arranged in a side-by-side arrangement.

6. The system of claim 1, wherein the needle section has a fluid injection lumen formed therein.

7. The system of claim 1, wherein the imaging device includes an intravascular ultrasound imaging transducer.

8. The system of claim 1, wherein the imaging device is slidably disposed within the injection catheter.

9. The system of claim 1, further comprising a third steerable catheter having a third lumen formed therein, wherein the first steerable catheter is disposed within the third lumen.

10. The system of claim 1, wherein the needle section includes a heating element.

11. The system of claim 1, wherein the needle section includes a cooling element.

12. The system of claim 1, wherein the needle section is configured to deliver electrocautery current to tissue.

13. The system of claim 1, wherein the needle section is configured to deliver a therapeutic gel to endocardial tissue.

14. A system for endocardial injection, the system comprising:
a steerable catheter assembly including two or more steerable catheters nested together;
an injection catheter disposed within the steerable catheter assembly, the injection catheter being configured to a therapeutic gel to endocardial tissue;
wherein the injection catheter has a distal end region with an imaging section and a needle section disposed adjacent to the imaging section;
an imaging device slidably disposed within the injection catheter; and
wherein the imaging device includes an ultrasound transducer configured to be disposed along the imaging section.

15. The system of claim 14, wherein the steerable catheter assembly includes at least one steerable catheter that is steerable in a single plane.

16. The system of claim 14, wherein the steerable catheter assembly includes at least one steerable catheter that is steerable in two planes.

17. The system of claim 14, wherein the needle section includes a heating element.

18. The system of claim 14, wherein the needle section includes a cooling element.

19. The system of claim 14, wherein the needle section is configured to deliver electrocautery current to tissue.

20. A method for delivering a therapeutic gel to endocardial tissue, the method comprising:
navigating a endocardial injection system through a body lumen to a target location, the endocardial injection system comprising:
a first steerable catheter having a first lumen formed therein,
a second steerable catheter disposed within the first lumen, the second steerable catheter having a second lumen formed therein,
an injection catheter disposed within the second lumen, the injection catheter having a distal end region,
wherein the distal end region includes an imaging section and a needle section, and
an imaging device configured to be disposed within the imaging section;
wherein navigating the endocardial injection system through the body lumen to the target location includes steering the first steerable catheter, steering the second steerable catheter, or steering both the first steerable catheter and the second steerable catheter;
engaging the target location with the needle section; and
delivering the therapeutic gel through the needle section and into the target location.
